(19) United States
(12) Patent Application Publication
(10) Pub. No.: US 2026/0158273 A1
(43) Pub. Date: Jun. 11, 2026

(54) METHOD AND SYSTEM FOR QUANTIFYING MOVEMENT PATTERNS IN A SUBJECT

(71) Applicant: Stardots AB, Uppsala (SE)

(72) Inventors: Daniel Petrini, Uppsala (SE); Emil Edin, Uppsala (SE); Alexander Medvedev, Täby (SE); Fredrik Olsson, Uppsala (SE)

(21) Appl. No.: 19/414,168

(22) Filed: Dec. 9, 2025

Related U.S. Application Data
(60) Provisional application No. 63/730,726, filed on Dec. 11, 2024.

(51) Int. Cl. A61N 1/36; A61N 1/372

(52) U.S. Cl. CPC A61N 1/36067; A61N 1/36139; A61N 1/37247

(57) ABSTRACT

A method and system for quantifying movement patterns in a subject includes receiving motion data representing voluntary and involuntary movements of the subject from a sensor device worn or held by the subject. A signal indicative of involuntary movement deviations occurring in a plane diverging from a voluntary movement trajectory is extracted and associated to a current position of the voluntary movement trajectory. Peaks within the extracted signal are identified, wherein each peak corresponds to a local maximum of involuntary movement magnitude. A probability density function (PDF) of the identified peaks is estimated and a symptom severity score is computed based on one or more quantiles of the estimated PDF. Feedback is provided to a user via a user interface based on the symptom severity score to adjust a parameter of a therapeutic device.

### CROSS REFERENCE TO RELATED APPLICATIONS

This application claims the benefit of U.S. Provisional Patent Application No. 63/730,726, filed Dec. 11, 2024, the contents of which are incorporated herein by reference in their entirety as if fully set forth.

### FIELD

Illustrative embodiments generally relate to medical diagnostics and treatment and, more particularly, various embodiments relate to using hand movement-based tests to diagnose and treat patients.

### BACKGROUND

Medical diagnostics and treatment often require testing with subjective criteria. For example, a medical professional may ask patients about the presence of various symptoms or ask patients to rate symptoms on an arbitrary scale.

### SUMMARY OF VARIOUS EMBODIMENTS

In accordance with one embodiment of the invention, a method for quantifying movement patterns in a subject includes receiving motion data representing voluntary and involuntary movements of the subject from a sensor device worn or held by the subject. A signal indicative of involuntary movement deviations occurring in a plane diverging from a voluntary movement trajectory is extracted and associated to a current position of the voluntary movement trajectory. Peaks within the extracted signal are identified, wherein each peak corresponds to a local maximum of involuntary movement magnitude. A probability density function (PDF) of the identified peaks is estimated and a symptom severity score is computed based on one or more quantiles of the estimated PDF. Feedback is provided to a user via a user interface based on the symptom severity score to adjust a parameter of a therapeutic device.

In some embodiments, the involuntary movement comprises at least one of tremor or dyskinesia.

In some embodiments, the PDF estimation includes using a kernel density estimation (KDE) process.

In some embodiments, the kernel density estimation uses a normal distribution as a kernel function.

In some embodiments, the kernel density estimation applies a bandwidth parameter determined by a maximum likelihood approach.

In some embodiments, the method includes truncating the estimated PDF.

In some embodiments, identifying peaks includes applying at least one of a threshold-based method, zero-crossing analysis, or derivative-based technique.

In some embodiments, the method includes extracting statistical features from the identified peaks, including at least one of minimum peak magnitude, maximum peak magnitude, mean peak magnitude, median peak magnitude, standard deviation, or variance.

In some embodiments, the extracted statistical features are utilized in at least one of a clinical decision support system, an adaptive therapeutic device, or a machine learning algorithm for classification or prediction.

In some embodiments, the method includes computing features from the identified peaks, wherein the features include at least one of a peak frequency, an inter-peak interval, or a peak clustering metric.

In accordance with one embodiment, a system includes a sensor configured to detect motion data representing voluntary and involuntary movements of a subject, and a processor in communication with the sensor. The processor is configured to receive, from the sensor, the motion data representing voluntary and involuntary movements of the subject, extract a signal indicative of involuntary movement deviations occurring in a plane diverging from a voluntary movement trajectory, associate the extracted signal to a current position of the voluntary movement trajectory, identify peaks within the extracted signal, wherein each peak corresponds to a local maximum of involuntary movement magnitude, estimate a probability density function (PDF) of the identified peaks, compute a symptom severity score based on one or more quantiles of the estimated PDF, and provide feedback to a user via a user interface based on the symptom severity score to adjust a parameter of a therapeutic device.

In some embodiments, the involuntary movement comprises at least one of tremor or dyskinesia.

In some embodiments, the PDF estimation includes using a kernel density estimation (KDE) process.

In some embodiments, the kernel density estimation uses a normal distribution as a kernel function.

In some embodiments, the processor is further configured to apply a bandwidth parameter determined by a maximum likelihood approach.

In some embodiments, the processor is further configured to truncate the estimated PDF.

In some embodiments, the processor is further configured to apply at least one of a threshold-based method, zero-crossing analysis, or derivative-based technique.

In some embodiments, the processor is further configured to extract statistical features from the identified peaks, including at least one of minimum peak magnitude, maximum peak magnitude, mean peak magnitude, median peak magnitude, standard deviation, or variance.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 schematically shows an example patient analysis system in accordance with various embodiments.

FIG. 2 shows a method for assessment of symptom severity a patient in accordance with various embodiments.

FIG. 3 shows an example graph showing peaks identified in a tremor signal in accordance with various embodiments.

FIG. 4 shows an example graph showing Kernel density estimation of a probability density function in accordance with various embodiments.

FIG. 5 shows an example graph showing a Bradykinesia signal with detected positive and negative peaks in accordance with various embodiments.

FIG. 6 shows an example test selection interface in accordance with various embodiments.

FIG. 7 shows an example calibration interface in accordance with various embodiments.

FIG. 8 shows an example test interface in accordance with various embodiments.

FIG. 9 is a graph showing an example accelerometer output during a calibration and test in accordance with various embodiments.

FIG. 10 is a graph showing an example orientation of the client device in accordance with various embodiments.

FIG. 11 illustrates an example projection of involuntary motion onto intended motion during a test in accordance with various embodiments.

FIG. 12 is an example graph showing projections of involuntary positions during a test in accordance with various embodiments.

FIG. 13 is an example graph showing the power spectral density of involuntary positions in accordance with various embodiments.

FIG. 14 is an example graph showing peaks of involuntary motions in accordance with various embodiments.

FIG. 15 is an example graph showing peak height probability of involuntary motions in accordance with various embodiments.

FIG. 16 is an example graph showing projected 2D positions and radii state in accordance with various embodiments.

FIG. 17 is an example graph showing severity scores in accordance with various embodiments.

FIGS. 18-19 show results interfaces in accordance with various embodiments.

FIG. 20 shows an example computing device in accordance with various embodiments.

### DESCRIPTION OF ILLUSTRATIVE EMBODIMENTS

In illustrative embodiments, a method and apparatus allow a user to operate a device to test a patient for one or more illnesses. Test measurements are quantified to provide data aiding in diagnoses. To that end, the method may operate a patient device with a movement sensor, conduct a movement test using the patient device, and determine, using the movement sensor, movement data corresponding to the movement test, determining voluntary motions using the movement data. The method includes determining radii of the involuntary motions, determining discrete states using the radii, determining a transition matrix using the discrete states. A stationary distribution using the transition matrix, and a severity score using the stationary distribution are determined. Details of illustrative embodiments are discussed below.

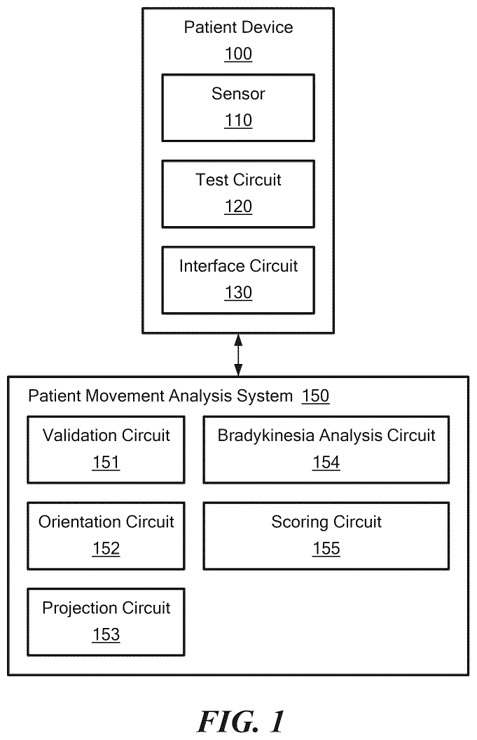

FIG. 1 schematically shows an example patient analysis system in accordance with various embodiments. In various embodiments, the patient analysis system includes a patient device 100 and a patient movement analysis system 150. The patient device is in communication with the patient analysis system and in various embodiments the communication may be wireless (e.g., IEEE 802.XX communication, cellular or otherwise) or a wired connection.

In various embodiments, the patient device includes a sensor 110, test circuit 120 and interface circuit 130. The sensor 110 may provide measurements to provide measurement data to the test circuit 120, which provides the measurement data to the interface circuit 130 for transmission to the patient movement analysis system 150. In various embodiments, the patient device may be referred to as a “mobile client device” that includes a mobile client (e.g., application) operating on it. In various embodiments, the sensor 110 may include an inertial or movement sensor, such as a gyroscope, accelerometer, or other sensor to determine movement of the patient device. Further, sensors may meet a sufficient quality rating with respect to various parameters (e.g., sampling rate, operation within expected ranges).

In various embodiments, the patient movement analysis system 150 includes a validation circuit 151 for validating received measurement data, an orientation circuit 152 for determining orientation of the patient device based on the measurement data, and a projection circuit 153 for projecting the measurement data analysis. The patient movement analysis system 150 also includes a bradykinesia circuit 154 for performing a bradykinesia analysis based on the measurement data and a scoring circuit 155 to determine scoring based on the measurement data. In various embodiments, the circuits described above may be in communication with one or more of the other circuits of the patient movement analysis system. In various embodiments, mobile application backend services and databases reside in and execute in the patient movement analysis system 150.

Although further detail is provided herein, in evaluation (e.g., for Parkinson's disease), medical diagnosis is graded on a scale relating to movement. In order to avoid the scale being too subjective, illustrative embodiments provide an objective method to assess patient movement, using current technology, such as a smart phone.

In various embodiments, the mobile client is responsible for guiding the user through tests, gathering sensor data during each test, displaying results to the user, and sending data to backend services for quantification.

The backend services preferably are responsible for handling the flow of data through internal systems. The method therefore may receive, by the backend services (e.g., patient movement analysis system 150) data from the mobile client (e.g., patient device 100), send raw measurements to a database 220 and to a quantification service 210, and then send results from the quantification service 210 to the database 220 and back to the mobile client 100.

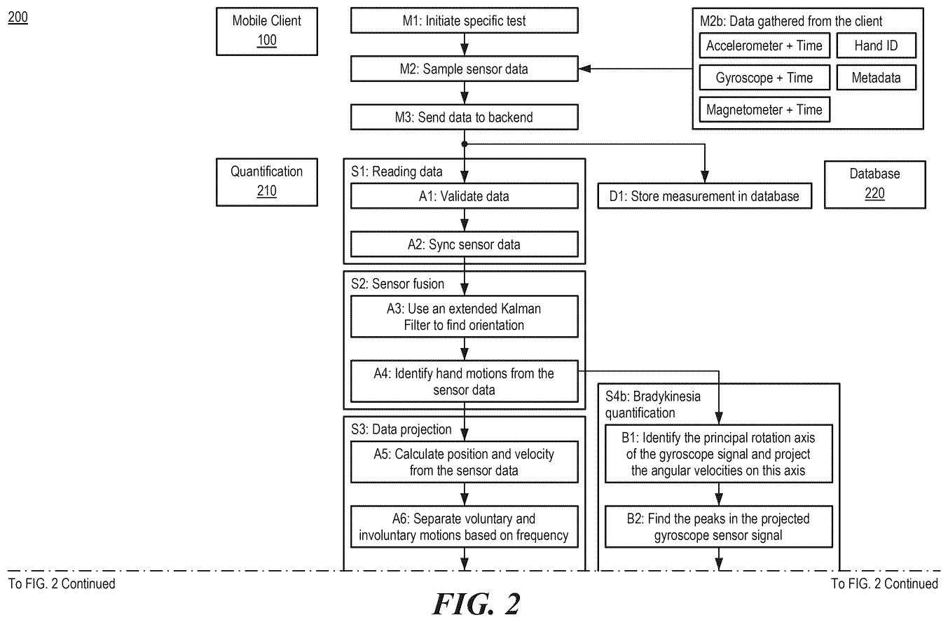

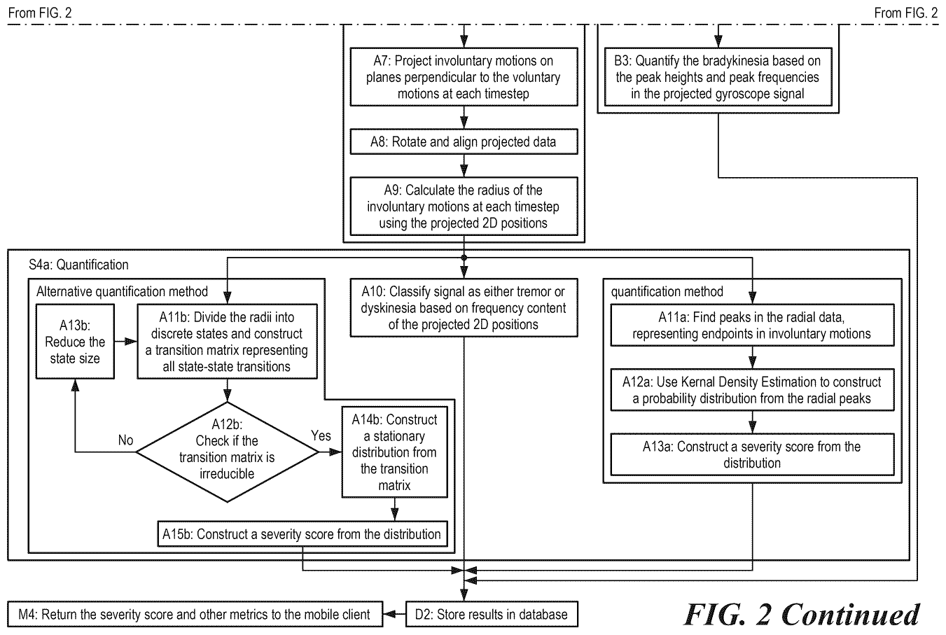

FIG. 2 is a flowchart showing an example method for assessment of symptom severity (e.g., symptom quantification) a patient in accordance with various embodiments. Although further detail is provided below, briefly, a quantification service is described herein where the algorithms for quantifying the sensor data resides. The algorithm can roughly be divided into 4 parts: I) receiving, validating, and syncing the sensor data, S1 in flowchart; II) calibrating the sensors and finding the orientation of the phone, S2 in flowchart; III) separating voluntary and involuntary motions and projecting the data to allow good comparisons, S3 in flowchart; IV) quantifying the severity of the involuntary motions, S4 in flowchart.

In various embodiments, the mobile client (e.g., patient device 100) may not perform the quantification, but may gather sensor data from the user.

Example tests that may be performed by a movement application residing on the mobile device may include, for example, a rest tremor test, a postural tremor test, an action tremor, a dyskinesia test, and a bradykinesia test. Tests can be selected individually or based on the user the tests can be bundled to force the patient to perform a series of tests in a particular order (e.g., for a clinical trial users can be set up to always perform the rest tremor, postural tremor, and action tremor tests in that order). For each test, the patient is asked to perform a certain motion or to hold the phone still in a particular manner for a specified period of time, for each test the instructions are repeated for both hands to allow a quantification to be made for each individual hand. Each of the above tests (which are described in further detail below) may be utilized to aid in diagnosis.

For example, rest tremors, a hallmark feature of Parkinson's disease (PD) and related parkinsonian syndromes, denote involuntary rhythmic oscillations that manifest primarily during a state of rest and are markedly attenuated or absent during voluntary movement. These tremors typically manifest as a low-frequency (4-6 Hz), pill-rolling movement of the hands, though they can also affect other body regions such as the legs, jaw, and tongue. Rest tremors are characterized by their amplitude modulation, whereby tremor intensity may wax and wane over time, often exacerbating during periods of stress or relaxation. The pathophysiology of rest tremors is intricately linked to the dysfunction of dopaminergic pathways within the basal ganglia, particularly involving the nigrostriatal system. Comprehensive understanding of the clinical features and underlying neurobiological mechanisms of rest tremors is crucial for accurate diagnosis and the development of targeted therapeutic strategies aimed at ameliorating symptomatology and improving patient outcomes.

Postural tremors, a common manifestation of essential tremor (ET) and Parkinson's disease (PD), represent involuntary rhythmic oscillations occurring during the maintenance of a static posture. These tremors primarily affect the upper extremities, though they can also involve the head, trunk, and lower limbs. Postural tremors are typically exacerbated by factors such as stress, fatigue, and caffeine consumption. Characterized by a frequency range of 4-12 Hz, postural tremors often present as a coarse, oscillatory movement that is distinguishable from other types of tremors, such as resting or kinetic tremors. The pathophysiology of postural tremors involves dysfunction within the central nervous system, implicating complex interactions between the basal ganglia, cerebellum, and their respective neural circuits. Understanding the clinical features and underlying mechanisms of postural tremors is paramount for accurate diagnosis and targeted therapeutic interventions, thereby enhancing patient management and quality of life.

Action tremors, a characteristic manifestation of various movement disorders including essential tremor (ET) and Parkinson's disease (PD), refer to involuntary rhythmic oscillations that emerge during the execution of voluntary movements. These tremors are distinct from rest tremors in that they are typically absent or significantly attenuated at rest and become more pronounced during activities such as reaching for objects, holding utensils, or performing fine motor tasks. Action tremors can exhibit a wide range of frequencies, from low-frequency (4-6 Hz) to higher frequencies (>10 Hz), and may present with different patterns including postural, kinetic, or intention tremors. The severity of action tremors may vary based on factors such as fatigue, stress, and medication status, often impairing activities of daily living and diminishing quality of life for affected individuals. The pathophysiology of action tremors involves complex interactions within the central nervous system, implicating aberrant signaling within the cerebello-thalamo-cortical and basal ganglia-thalamo-cortical circuits. A comprehensive understanding of the clinical characteristics and underlying neurobiological mechanisms of action tremors is essential for accurate diagnosis and the development of targeted therapeutic interventions aimed at alleviating symptomatology and enhancing functional independence.

Dyskinesia, a multifaceted motor phenomenon commonly observed in individuals with Parkinson's disease (PD) and other movement disorders, encompasses a spectrum of involuntary, abnormal, and often complex movements that manifest as hyperkinetic fluctuations in muscle tone and activity. These movements can range from chorea, characterized by rapid, irregular, and unpredictable muscle contractions, to dystonia, involving sustained or intermittent muscle contractions resulting in abnormal postures or twisting movements. Dyskinesia typically emerges as a complication of long-term levodopa therapy in PD, with peak-dose dyskinesia occurring during periods of medication efficacy and off-period dystonia arising as levodopa levels decline. The pathophysiology of dyskinesia is intricately linked to dopaminergic denervation and pulsatile stimulation of striatal dopamine receptors, resulting in maladaptive neuroplastic changes within the basal ganglia-thalamo-cortical circuitry. Comprehensive understanding of the clinical manifestations and underlying neurobiological mechanisms of dyskinesia is crucial for optimizing treatment strategies aimed at mitigating its impact on motor function and improving the overall quality of life for affected individuals.

Bradykinesia, a cardinal feature of Parkinson's disease (PD) and related movement disorders, denotes a characteristic slowness of voluntary movements that is often accompanied by a reduction in the amplitude and force of motion. It manifests across various motor tasks, including gait initiation, repetitive movements, and fine motor control, resulting in impaired motor coordination and efficiency. Bradykinesia typically worsens over time and can significantly impact activities of daily living, leading to difficulties in tasks such as writing, dressing, and swallowing. The pathophysiology of bradykinesia involves dysfunction within the basal ganglia-thalamo-cortical circuitry, particularly affecting dopaminergic projections from the substantia nigra pars compacta to the striatum. Comprehensive assessment of bradykinesia is essential for accurate diagnosis and treatment optimization, as it serves as a key determinant of disease severity and progression in PD and related disorders.

Accordingly, testing for the above is useful for assessment, and/or diagnosing a patient, and therefore, in various embodiments, the tests may be performed in accordance with the following:
Rest tremor test: The test mimics the rest tremor test in the MDS-UPDRS protocol but is carried out with the patient holding the phone. In various embodiments, an elastic strap may be used to secure the phone to each respective hand to allow for measuring during a relaxed state, requiring no intentional gripping of the phone required by the patient. The patient is asked to calibrate the phone and then to place it in the selected hand. While sitting down the patient holds the phone in a relaxed manner with both hands or lower arms resting on their lap and palms facing upwards for 10 seconds. The patient is then asked to switch hands and perform the same resting motion with the other hand.
Postural tremor test: The test mimics the postural tremor test in the MDS-UPDRS protocol but is carried out with the patient holding the phone. The patient is asked to calibrate the phone and then to place it in the selected hand. While sitting down the patient holds the phone with arms stretched out in front of their body with palms facing down for 10 seconds. The patient is then asked to switch hands and perform the same test motion with the other hand.
Action tremor test: The test mimics the action tremor test in the MDS-UPDRS protocol but is carried out with the patient holding the phone. The patient is asked to calibrate the phone and then to place it in the selected hand. While sitting down the patient holds out their arms in front of their body with palms facing down and holding the phone in the specified hand. The patient then moves the arm holding the phone from the outstretched position slowly until the tip of the phone gently touches the tip of the patients' nose, this motion is repeated 3 times. The patient then switches the phone to the other hand and repeats the test motion with the other hand. In various embodiments, smoothness of motion is targeted for detection.
Dyskinesia test: The patient is asked to calibrate the phone and then to place it in the selected hand. While sitting down the patient puts the hand holding the phone straight down and keeps it down for 10 seconds. When the 10 seconds are done the patient raises their arm from the down stretched position and lifts it to their ear as if answering a phone call and then lowers it to about waist height. The lifting motion is repeated 3 times. Then the patient switches the phone to the other hand and starts off holding the phone straight down for 10 seconds before performing the same 3 lifts as for the first hand.
Bradykinesia test: The test mimics the bradykinesia test in the MDS-UPDRS protocol but is carried out with the patient holding the phone. The patient is asked to calibrate the phone and then to place it the selected hand. While sitting down the patient holds the phone with arms stretched out in front of their body with palms facing down. The patient then turns the hand holding the phone so that the palm faces upward. This motion is repeated back and forth for 10 seconds with the patient attempting to turn the hand holding the phone as quickly back and forth as possible with as large an angle as possible. After 10 seconds the patient is asked to switch hands and the test motion is repeated for the second hand.

Each test starts with a short calibration phase where the user is asked to put the phone down on a still surface to find a stable initial direction, then the patient is asked to place the phone in the selected hand and start the test. Once the test for the first hand is carried out, the patient is asked to place the phone in the other hand and perform the test for that hand as well and finally end the test.

The data from the test is then sent to on-premise or cloud servers where the backend stores the raw data in the database and sends the data to the quantification backend service. The data from a particular test is sent to the corresponding quantification service endpoint where each test has its own separate function responsible for the quantification.

Accordingly, FIG. 2 shows a mobile client, quantification function/service and database in accordance with various embodiments.

At block M1, a specific test (e.g., as described above) may be initiated. At block M2, sensor data is sampled. For example, at block M2b, data such as accelerometer and time, gyroscope and time, magnetometer and time, hand ID, and/or metadata is gathered from the client. In various embodiments, the data may be gathered in accordance with the tests described above.

Accordingly, at block M3, the data is sent/transmitted to the backend. At block D1, the measurement data sampled is stored in the database. At block A1, the data is validated and at block A2, the sensor data is synchronized. Collectively, blocks A1 and A2 may be considered a reading data block S1.

At block A3, a filter (e.g., extended Kalman filter) is utilized to find orientation, and at block A4, hand motions from the sensor data (e.g., measured data) is identified. In various embodiments, incorrect motion information may be flagged and the user may be prompted to redo the test. Collectively, blocks A3 and A4 may be considered a sensor function block S2.

From block A4, the method proceeds to either block A5 or block B1.

For example, at block A5, position and velocity is calculated from the sensor data. At block A6, voluntary and involuntary motions based on frequency are separated. At block A7, involuntary motions are projected on planes perpendicular to voluntary motions at each timestep. In various embodiments, the voluntary motion projection radius may correspond to the magnitude of the motion.

At block A8, the projected data is rotated and aligned, and at block A9, the radius of the involuntary motions at each timestep is calculated using projected two dimensional (2D) positions. Collectively, blocks A5-A9 may be considered a data projection block S3.

As shown in FIG. 2, there are two quantification blocks S4a and S4b. Quantification block S4b may be a Bradykinesia quantification block.

Referring now to block S4a, at block A10, a signal is classified as either a tremor or dyskinesia based on a frequency content of the projected 2D positions. In various embodiments a quantification method (e.g., blocks A11a, A12a and A13a) are performed to quantify the data in a first quantification method. In various embodiments, a second quantification method (e.g., alternative quantification method) may be performed in blocks A11b, A12b, A13b, A14b and A15b.

In various embodiments, block S4a is responsible for the modeling and quantification of a signal extracted from involuntary movement patterns, for example tremor, hyperkinesia or dyskinesia. This signal, which may be referred to as the tremor signal, captures deviations in movement that occur in a plane orthogonal to the direction of a voluntary motion—essentially representing unintended side-to-side or up-and-down fluctuations that accompany purposeful actions.

The tremor signal is dynamically anchored (e.g., associated) to the current position of the voluntary movement trajectory, meaning its origin shifts in real time as the individual moves. This ensures that the tremor is measured relative to the intended motion path, allowing for more accurate characterization of the disorder.

The magnitude of the tremor signal may serve as a quantitative descriptor of the disorder's severity. In various embodiments, it reflects the amplitude of the involuntary deviations, which can be interpreted as the intensity or impact of the underlying neurological condition. By analyzing this magnitude, clinicians or algorithms can assess the progression of the disorder, evaluate treatment efficacy, or tailor therapeutic interventions.

Referring now to block A11a, peaks are identified within a tremor signal (e.g., the local maxima of the signal's magnitude). These peaks correspond to moments when the involuntary movement reaches its highest intensity, and they serve as indicators of the amplitude and severity of the underlying movement disorder, such as tremor or dyskinesia.

In various embodiments, the tremor signal is derived from motion data that isolates involuntary deviations from voluntary movement. The signal is typically processed to remove noise and baseline drift, allowing for accurate detection of peak events. Once the signal is stabilized, peak detection algorithms—such as threshold-based methods, zero-crossing analysis, or derivative-based techniques—are applied to identify significant excursions in the signal magnitude.

Each detected peak is characterized by its magnitude (i.e., how severe the tremor is at that moment) and its temporal location (i.e., when it occurred). These values are stored in a structured list or array for subsequent statistical analysis.

From this list of peaks, one or more of the following statistical features are extracted to quantify the tremor profile:

Minimum Peak Magnitude: Represents the lowest observed tremor amplitude, useful for establishing baseline activity.

Maximum Peak Magnitude: Indicates the highest observed tremor amplitude, reflecting the most severe symptom manifestation.

Mean Peak Magnitude: Provides an average measure of tremor intensity across the observation period.

Median Peak Magnitude: Offers a robust central tendency metric, less sensitive to outliers than the mean.

Standard Deviation of Peak Magnitudes: Quantifies the variability in tremor intensity, useful for assessing symptom consistency.

Variance of Peak Magnitudes: A complementary measure of dispersion, often used in machine learning models and statistical classifiers.

In various embodiments, the above features may be utilized in clinical decision support systems, where they inform diagnosis, treatment planning, and monitoring of disease progression. They may be also used in adaptive therapeutic devices, which adjust stimulation parameters based on real-time tremor severity. In addition, they may be used in machine learning algorithms, which classify movement disorder types or predict symptom trajectories based on statistical patterns.

In some embodiments, additional features such as peak frequency, inter-peak intervals, and peak clustering metrics may also be computed to enhance the granularity of tremor characterization.

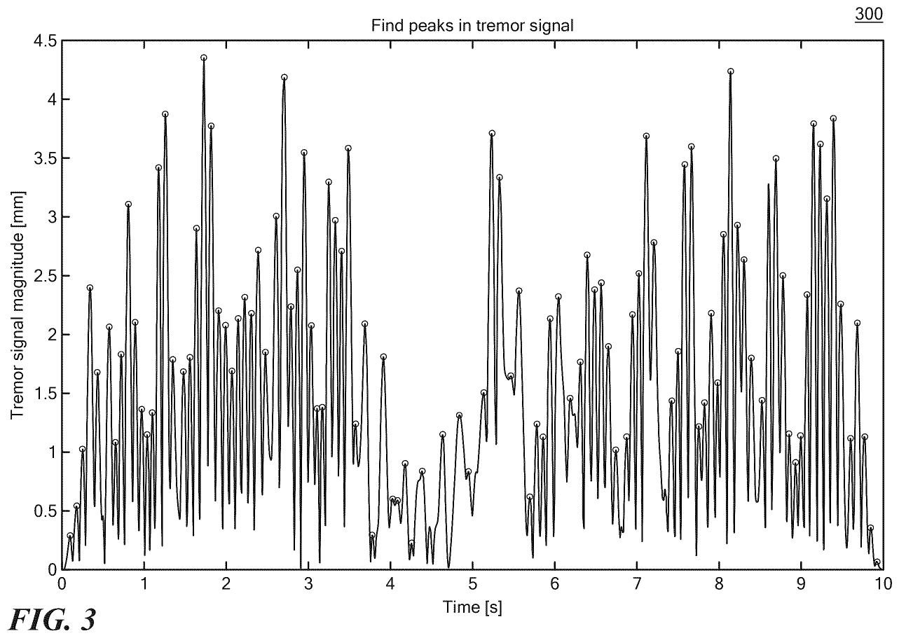

FIG. 3 shows an example graph 300 showing peaks identified in a tremor signal in accordance with various embodiments.

At block A12a, Kernel density estimation is used to construct a probability distribution from the radial peaks.

In various embodiments, lists of identified tremor signal peaks serve as a primary input for the kernel density estimation (KDE) process. KDE is a non-parametric technique widely used for estimating the probability density function (PDF) of a random variable based on observed data. The underlying distribution is approximated by summing a set of kernel functions, each centered at an observed data point, thereby producing a smooth estimate of the PDF.

Several parameters may govern the behavior of the KDE:

Kernel Function: Defines the shape of the contribution from each data point. In our case, we use a normal (Gaussian) distribution as the kernel, which ensures smooth and symmetric contributions around each peak.

Bandwidth: Acts as a smoothing parameter. A smaller bandwidth may result in a PDF that closely follows the individual peaks, potentially capturing fine details but also may introduce noise. Conversely, a larger bandwidth produces a smoother PDF by spreading each kernel more broadly, reducing variance but potentially oversmoothing the data.

The optimal bandwidth and other KDE parameters are determined using, for example, a maximum likelihood approach, which selects the values that maximize the likelihood of observing the given data under the estimated density model.

Focusing the analysis on tremor amplitude, the following example constraints may be imposed on the support of the estimated PDF:

Lower Bound: Exclude negative values because amplitude cannot be negative.

Upper Bound: To avoid computational inefficiencies and unrealistic infinite support, the PDF may be truncated, for example, at (0, (max (peaks)+bandwidth)×1.25).

This ensures the range covers all observed peaks plus a margin for smoothing, while remaining finite. The truncation step is introduced to attempt to ensure that the estimated density is both physically meaningful and computationally manageable.

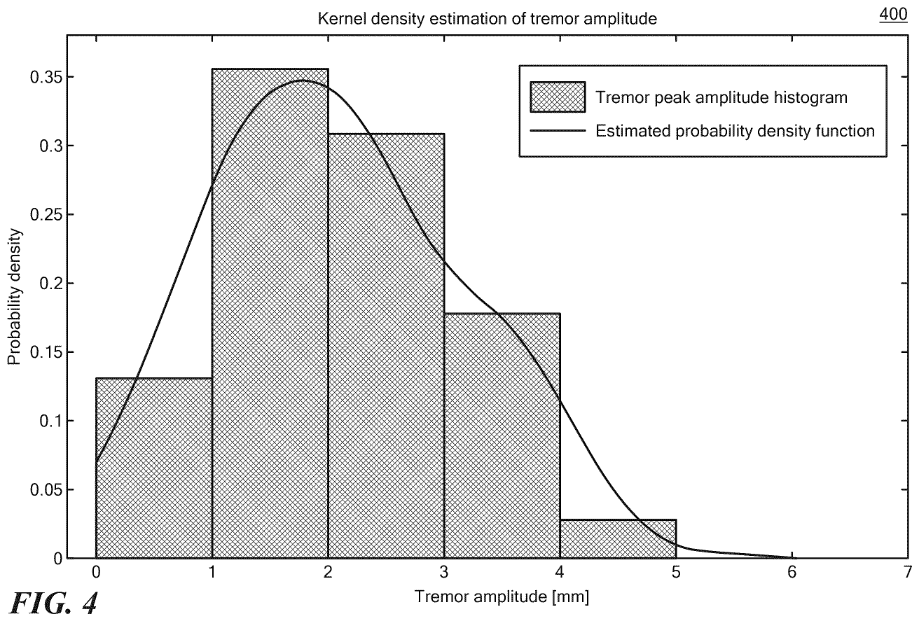

FIG. 4 shows an example graph 400 showing Kernel density estimation of a probability density function in accordance with various embodiments, As shown in FIG. 4, a histogram of the input tremor signal peaks is shown in the step blocks and the corresponding PDF estimated using the KDE technique is shown by the associated curve.

At block A13a, a severity score is constructed from the distribution.

In various embodiments, the estimated probability density functions (PDFs) derived from the tremor signal are used to compute a symptom severity score, which provides a quantitative measure of tremor intensity for a specific hand. The severity score may be defined in accordance with the following equation:

$$score = 100 tanh ( ( {q}_{50} ( p ) + 1.4 {q}_{75} ( p ) + {q}_{95} ( p ) + {q}_{100} ( p ) ) \frac{2 π}{0.9} ) ,$$

where:
p: The estimated PDF of tremor amplitude for one hand.
qₖ(p): The k-th percentile (quantile) of the distribution, representing the amplitude value below which k % of the observations fall.
q₅₀: Median amplitude.
q₇₅: Upper quartile (captures moderate tremor severity).
q₉₅ and q₁₀₀: Extreme values (captures severe tremor episodes).
tan h: The hyperbolic tangent function, which compresses the output into a bounded range, ensuring the score remains between 0 and 100.

By weighting the sum of quantiles, higher amplitude values may be emphasized, which may be more indicative of severe tremor. The factor 1.4 applied to q₇₅ may provide extra weight to moderate tremor levels, improving sensitivity in early stages. Multiplying by 2π/0.9 may scale the combined quantile sum before applying the non-linear transformation. Further the tanh function may be utilized to introduce saturation. For example, as tremor severity increases, the score approaches 100 but never exceeds it. This may serve to prevent extreme outliers from disproportionately inflating the score.

For mild tremor signals, the score may increase gradually, providing fine granularity for small to moderate symptom severities. For severe tremor signals, the score may rise quickly and then levels off near 100, reflecting a plateau in symptom severity classification.

In another (alternate) quantification method, at block A11b, radii are dividing into discrete states and a transition matrix representing all state-state transitions is constructed. At block A12b, it is determined/checked if the transition matrix is irreducible. If at block A12b, the transition matrix is not irreducible, then the method proceeds to block A13b, where the state size is reduced. However, if, at block A12b the transition matrix is irreducible, then the method proceeds to block A14b, where a stationary distribution is constructed from the transition matrix, and at block A15b, a severity score is constructed from the distribution.

Referring back to block A4, if a bradykinesia quantification is to performed, then the method proceeds to block B1, wherein the main frequency of the gyroscope sensor signal is identified. At block B2, the peaks in the gyroscope sensor signal are found and at block B3, the bradykinesia is quantified based on the peak highs and frequencies (e.g., in Hz) in the gyroscope signal. Collectively, blocks B1-B3 may be referred to as a bradykinesia quantification block S4b.

In various combinations, bradykinesia quantification block S4b handles modeling and quantification of an extracted bradykinesia signal. That is, the bradykinesia block is responsible for modeling and quantifying the extracted bradykinesia signal, which reflects the subject's ability to perform rapid, repetitive rotational movements. This signal is derived from angular velocity measurements captured during the bradykinesia test. In various embodiments of this test, the subject is instructed to:
1. Hold the smartphone firmly in one hand.
2. Extend both arms straight out in front of the body.
3. Rotate the hand holding the phone back and forth—pronation and supination—as fast and as far as possible for a duration of 10 seconds.
4. Repeat the procedure for the other hand.

The resulting bradykinesia signal captures the dynamic characteristics of these rotations. Example key clinical markers of bradykinesia include progressive slowing of rotational speed (e.g., as the test continues, individuals with bradykinesia often exhibit a noticeable decline in angular velocity, reflecting impaired motor initiation and execution), and a reduction in rotation amplitude (e.g., successive movements tend to become smaller in range, indicating diminished movement vigor and flexibility).

By analyzing these patterns (e.g., the temporal decay in speed and the amplitude reduction), the bradykinesia block provides a quantitative representation of symptom severity. This enables objective assessment of motor impairment for monitoring disease progression and evaluating treatment response.

For example, block B1 a gyroscope signal recorded during each hand movement in the bradykinesia test undergoes a series of preprocessing steps to ensure accurate extraction of the bradykinesia signal. These steps are directed to reduce noise, remove irrelevant trends, and isolate the dominant rotational component of the movement.

For example, smoothing using a cubic Savitzky-Golay filter with a filter window corresponding to 150 milliseconds performs local polynomial regression (in this case, cubic) within the window to smooth the signal while preserving important features such as peaks and slopes.

Detrending may be utilized to remove a cubic polynomial trend of the signal. That is, a smoothed signal often contains slow-varying trends unrelated to the actual rotational movement. To remove these, a cubic polynomial trend is fitted and subtracted from the signal.

A centered principal component analysis (PCA) is performed on the 3-axis gyroscope data. The PCA identifies the directions of maximum variance in the signal. For rotational movements, the first principal component typically corresponds to the principal axis of rotation.

The original gyroscope signal is projected onto the identified principal axis. This projection isolates the component of angular velocity that represents the actual rotational movement, removing contributions from secondary or unintended motions. Accordingly, the resulting one-dimensional signal may be referred to as the bradykinesia signal, which serves as the basis for quantifying symptom severity.

At block B2, to estimate the rotational frequency of the movement, the following computation is performed:

$$peakFrequencies = \frac{sampleRate}{peakToPeakSampleDistance}$$

where the following apply:
sampleRate: The sampling frequency of the gyroscope sensor, expressed in Hertz (Hz).
peakToPeakSampleDistance: The number of samples between two successive peaks in the concatenated sequence.

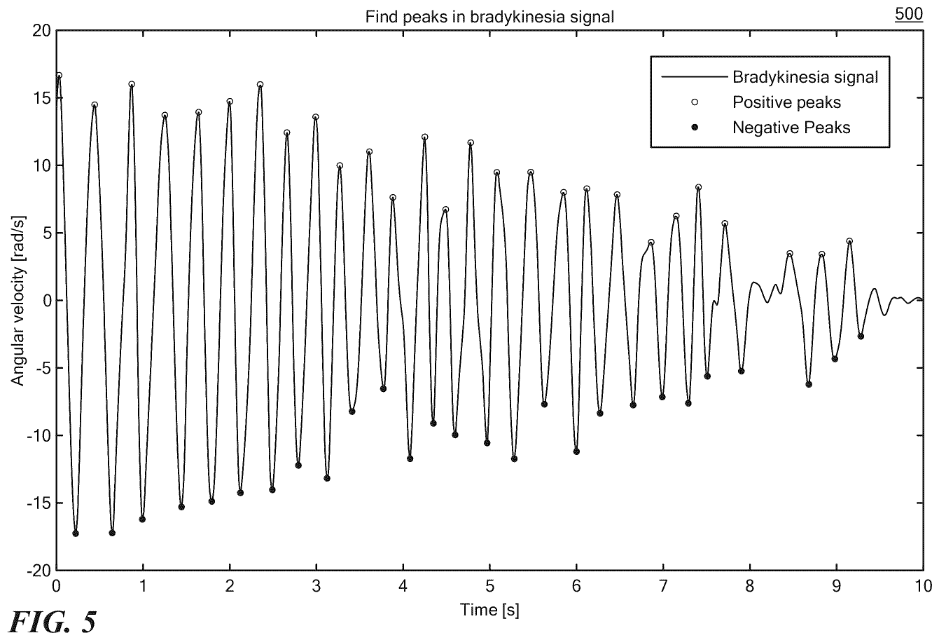

FIG. 5 shows an example graph 500 showing a Bradykinesia signal with detected positive and negative peaks in accordance with various embodiments.

At block B3, a bradykinesia severity score is computed based on peak data extracted from the processed bradykinesia signal. This score provides an objective measure of motor impairment severity during the rotational movement test. The score may be defined in accordance with the following equation:

$$score = min ( \frac{0.5 peakIQR + freqIQR}{peakMedian + 3 freqMedian} * 100 , 100 )$$

where the following apply:
peakIQR: Interquartile range of peak heights (amplitude of rotational movements).
freqIQR: Interquartile range of peak frequencies (rate of rotational movements).
peakMedian: Median of peak heights.
freqMedian: Median of peak frequencies.
min(⋅, 100): Saturates the score at a maximum value of 100 to prevent outlier-driven inflation.

In various embodiments, a min function is used to saturate any score value to a maximum of 100. Larger interquartile ranges are indicative of inconsistencies in the rotational movements observed in bradykinesia symptoms whereas larger peak medians are indicative of faster and larger movements. For more severe bradykinesia symptoms the numerator of the score is expected to increase whereas the denominator is expected to decrease, causing the score to increase significantly for more severe symptoms.

From blocks A10, A13a, A15b or B3, results are stored in the database at block D2 and the severity score and other metrics are returned to the client at block M4. In various embodiments, this may include wirelessly transmitting the data to the patient device described in FIG. 1. In various embodiments, the severity score may be a measure of current state, could track progression, and/or could cause an adjustment in treatment, such as medication or deep brain stimulation.

In order for a patient to perform the tests and see results, visual representations may be utilized on the screen of the patient to device to guide him or her as to how to perform the test or to display results.

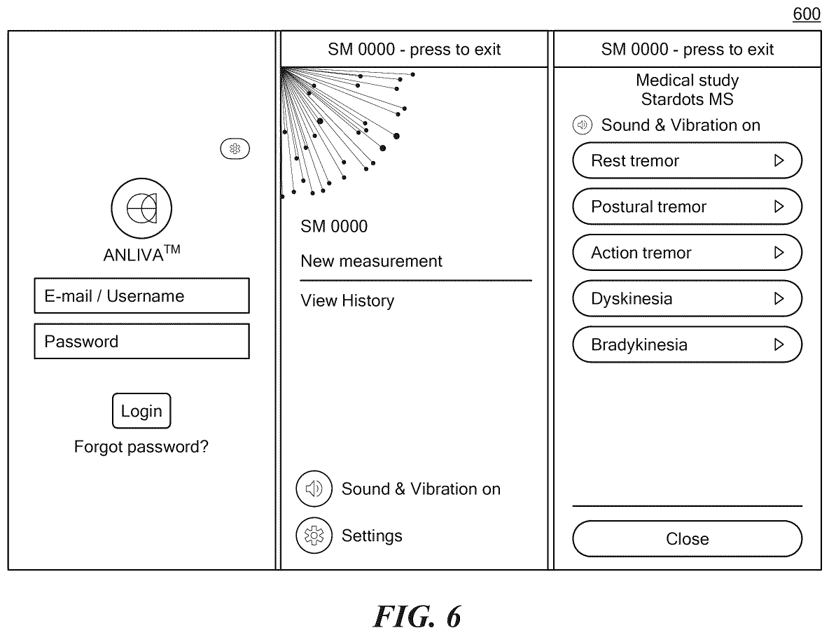

FIG. 6 shows an example test selection interface 600 in accordance with various embodiments. FIG. 6 shows example base screens that the patient may encounter when opening the application, after logging in, and choosing a measurement to perform. For example, as shown in the screen to the right of FIG. 6, test types for selection are shown.

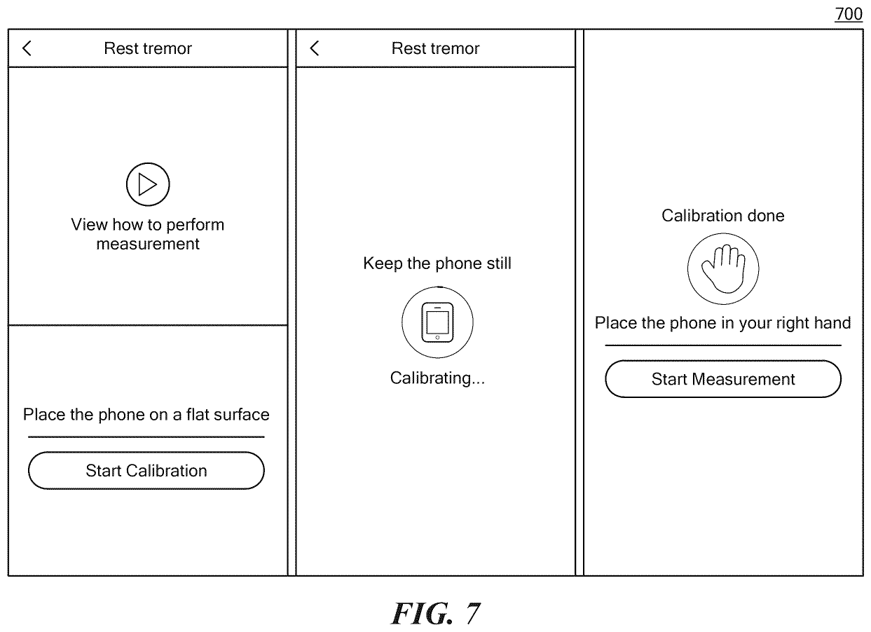

FIG. 7 shows an example calibration interface 700 in accordance with various embodiments. For example, FIG. 7 shows the screen as shown to the user after selecting a particular measurement to perform. In various embodiments, the patient is met by the calibration screen which allows for an instructional video to be watched, then the calibration is performed at the initiation of the patient. The calibration continues until the sensors in the phone registers a section in time of correct length where the phone has been still enough. When the calibration is done, the patient is asked to place the phone in the specified hand and initiate the measurement part of the test.

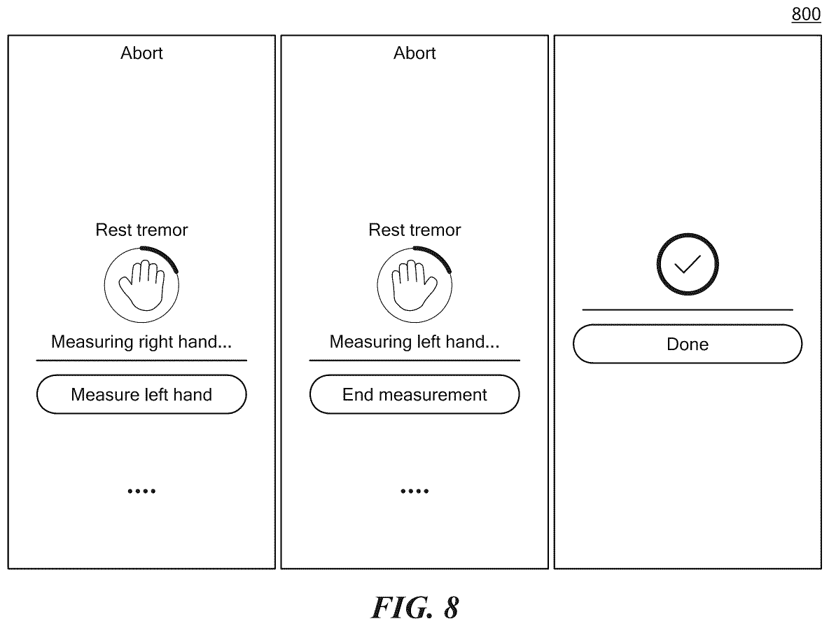

FIG. 8 shows an example test interface 800 in accordance with various embodiments. For example, in various embodiments, FIG. 8 shows example measurement screens as seen by the patient after the calibration phase is done. The patient performs the intended motion for the first selected hand, then clicks the button to measure the other hand in the same way and finally ends the test.

Further description of various blocks of FIG. 2 are described herein. For example, referring again to block M1, the user selects a specific test to perform, or the client prompts the user to perform a specific test. The test allows the user to view an instructional video of the test and all tests then consists of a short calibration phase which starts at the press of a button from the user. Once done, the user is asked to place the phone in the correct hand and the test will start at the press of a button from the user. The user performs the motion associated with the test and then places the phone in the other hand. The test for the other hand will start at the press of a button from the user. Once done, the user ends the test by pressing a button.

Referring again to block M2, the calibration phase of the test is started the accelerometer, gyroscope, and magnetometer samples measurements at the specified sample frequency, set at a 100 Hz, and stores the sensor readings along with a timestamp for each reading in 3 4D arrays (time, x, y, z). The sensor readings do not stop until the user is finished with the test or aborts the test. Along with the sensor readings an index parameter is also stored indicating if the test is in the calibration phase, the measurement phase for the first hand or the measurement phase for the second hand. Metadata about the system and the measurement session is also saved. During the calibration phase the measurements are monitored to make sure that the phone is sufficiently still so that it will provide a good initial calibration and orientation estimate. If the phone is moving too much the calibration time indicator will keep resetting and starting over until a long enough sequence of stationary measurements have been sampled.

Referring again to block M2b, the data is gathered during the test.

Referring again to block M3, once a measurement is done the data is uploaded to the backend for processing, and referring again to block M4, once a test is completed and the quantification performed in the backend, the data is retrieved from the result database for display in the client.

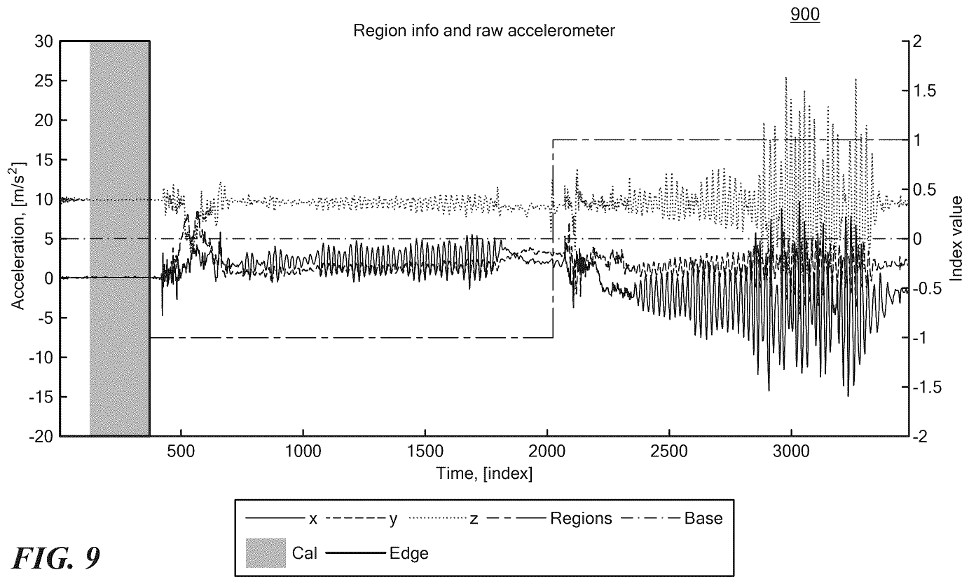

FIG. 9 is a graph 900 showing an example accelerometer output during a calibration and test in accordance with various embodiments. For example, described above in FIG. 2, block S1 handles the incoming data, validates it, and syncs the sensor measurements. Referring to block A1 above, once the test data arrives to the specified quantification endpoint it contains 3 fields of 4D sensor data (time, x, y, z) from the mobile phone accelerometer, gyroscope, and magnetometer as well as a 1D index vector indicating if the corresponding sensor data comes from either calibration, hand 1 or hand 2 (as shown in FIG. 9). The incoming data can also contain metadata (e.g. timestamp of the test, phone model, sensor package model etc.). The data is validated to make sure that it has the correct format and that the time details for the sensors have been sampled at an appropriate frequency and without any interruptions. In various embodiments, 100 Hz may be utilized as a sampling time in the mobile client while allowing sampling as low as 50 Hz in the backend and with single interruptions of 1/10 second.

Referring again to block A2, if the time details are within specification the sensor data is synced to give sensor readings at the same timesteps for all sensors. This is done by finding the first and last measurement timestep within which all sensors have some measurement (e.g., the accelerometer and the gyroscope might have 3 samplings before the magnetometer has made its first sampling and then the magnetometer might have its last measurement 2 timesteps after the accelerometer and gyroscope has their last sampling while all having a valid frequency). Then the data to use is selected as the span from the first magnetometer reading to the last accelerometer and gyroscope reading. After this selection has been made, the selected data is then interpolated to sync the timesteps of all sensors.

Referring back to block S2 above, the block handles the calibration and sensor fusion to find the orientation of the phone using the accelerometer, gyroscope, and the magnetometer. It also handles the selection of the specific hand sections of interest. Here the algorithm differs slightly between the rest tremor, postural tremor, and bradykinesia tests on the one hand and the action tremor and dyskinesia test on the other. The first set of tests are time based (i.e., the patient performs the test for a specified period of time), while the second set are motion based where the patients perform a particular motion.

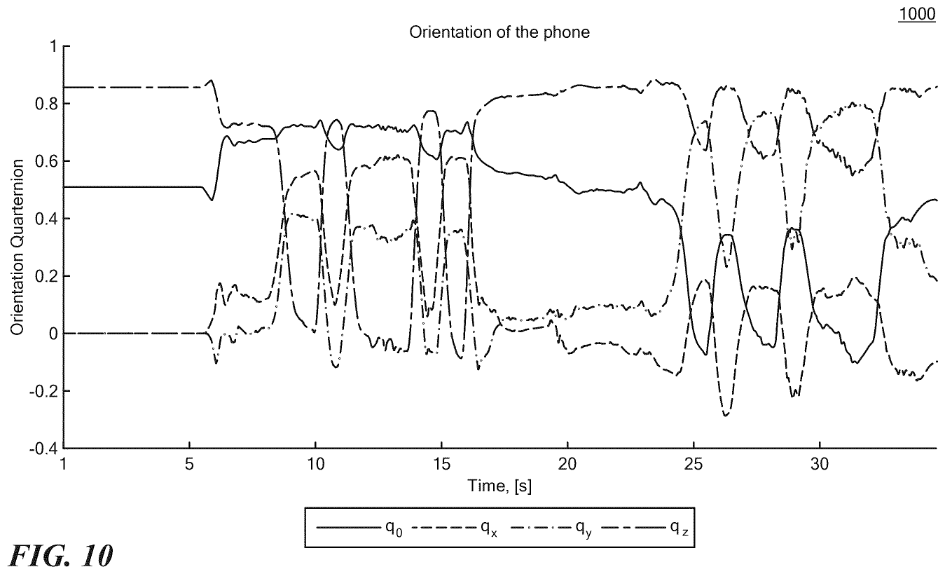

FIG. 10 is a graph 1000 showing an example orientation of the client device in accordance with various embodiments. Referring back to block A3, to find the orientation of the phone an implementation of an extended Kalman filter is used fusing the data from the accelerometer, gyroscope, and magnetometer together to get a good description of the orientation in quaternion form.

Referring again to block A4, the orientation is found the sections in the data representing the motions or sections of interest are picked out. For the time-based tests, rest, postural, and bradykinesia the sections are simply picked out based on the test times for each test. For the motion-based tests, the data is picked out by identifying the repeating patterns of the specific motions from the sensor signals.

Referring again to block S3 the separation of the involuntary and voluntary motions present in the test data and the projection of involuntary motions to allow good comparisons to be made is described. This section, in various embodiments, applies to the tests for rest tremor, postural tremor, action tremor, and dyskinesia, not to the bradykinesia test.

Referring back to block A5, the accelerometer signal is integrated twice to provide both velocity and position of the phone during the test. The orientation is used to remove the contribution of gravity on the accelerometer signal.

Referring again to block A6, a filter (e.g., Savitsky-Golay filter) with appropriate window length is used to filter the data and divide it up into a voluntary part (the low frequency data) and an involuntary part (the high frequency data). For the movement-based tests, (i.e. action tremor and dyskinesia test, the window length represents a trade off between keeping lower frequency dyskinetic movements in the involuntary movement signal and removing the test motion that the patient is asked to perform). Therefore, the window length must be chosen with care based on the minimum specified length set for a valid test motion. For the stationary tests, (i.e. rest tremor and postural tremor), this may be less important as no voluntary motions are to be performed during the tests, here the window length is set to a larger value to simply remove any very low frequency motions that might possibly occur such as slight shifting of position or small postural motions during the tests.

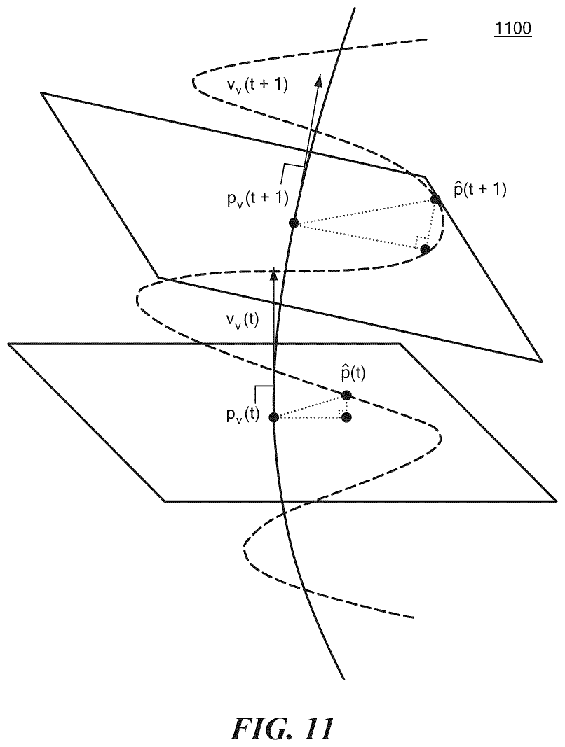

Referring then again to block A7, after the motions have been separated into voluntary and involuntary parts the gradient of the voluntary motion is calculated and used at each timestep to construct a plane which is perpendicular to the voluntary path, (i.e. it has the velocity of the voluntary motion as its normal vector). At each timestep the corresponding involuntary motion is then projected onto the constructed plane. FIG. 11 illustrates an example projection of involuntary motion onto intended motion during a test in accordance with various embodiments.

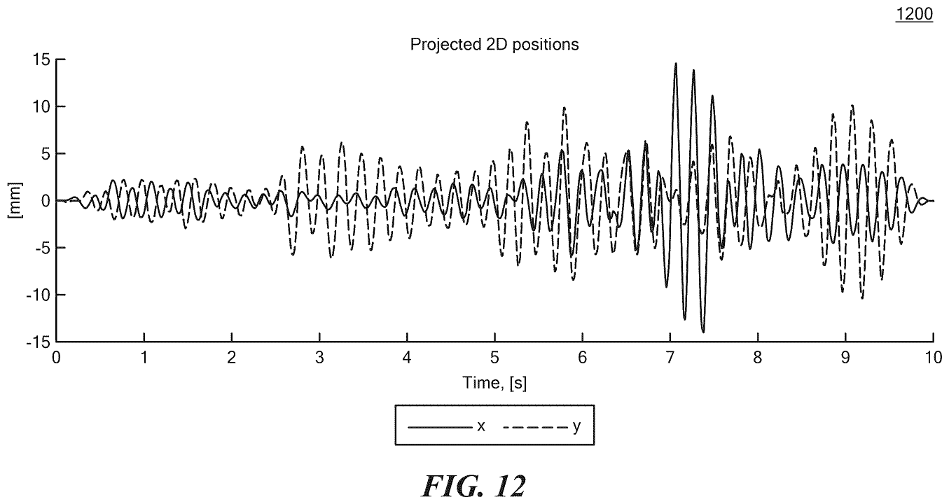

FIG. 12 is an example graph 1200 showing projections of involuntary positions during a test in accordance with various embodiments. Referring again to block A8, once the projections are done, the first plane, along with the projected involuntary motion, is rotated to align it with the second plane, next the first two planes are rotated to align both with the third plane and so on until all planes are aligned. Finally, the x and y components of the involuntary motions are picked out, (see FIG. 12), discarding the z component as there is a lot of uncertainty in the separation of the involuntary motion from the voluntary motion in the direction of the voluntary motion.

Referring again to block A9, the radii of the involuntary motions are calculated by taking the norm of the projected 2D positions at each point in time. In various embodiments, this may represent the magnitude, or the amplitude, of the involuntary motions.

Block S4, as described above, describes the quantification part of the algorithms. In various embodiments, there are two versions performed with, the first model that picks out the radii peaks and constructs a probability distribution describing the probability of specific involuntary motions occurring during the test. The other one describes the involuntary motions as a Markov chain and calculates a distribution describing the probability of the phone being in a specific radial state (discrete radii level) at any given time during the test. It is the first of these that is described at blocks A11a-A13a, while the second quantification version is described under blocks A11b-A15b.

Referring to block A10 again, from the projected 2D involuntary positions the power spectral density is calculated using Welch's method of averaging overlapping segments, see FIG. 10. This provides an estimate of the frequency content of the involuntary motions. This is used to provide a classification of the type of involuntary motions that are present during the test. A larger lower frequency component, around 2 Hz but up to 3.5 Hz, will lead to a classification of the involuntary movements as dyskinetic movements, while a larger frequency component in the range above 3.5 Hz will lead to a tremor classification.

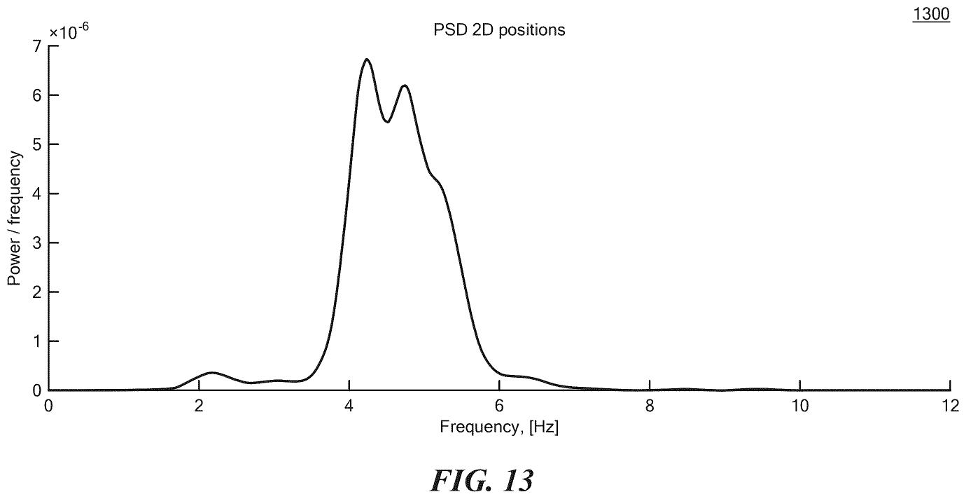

FIG. 13 is an example graph 1300 showing the power spectral density of involuntary positions in accordance with various embodiments.

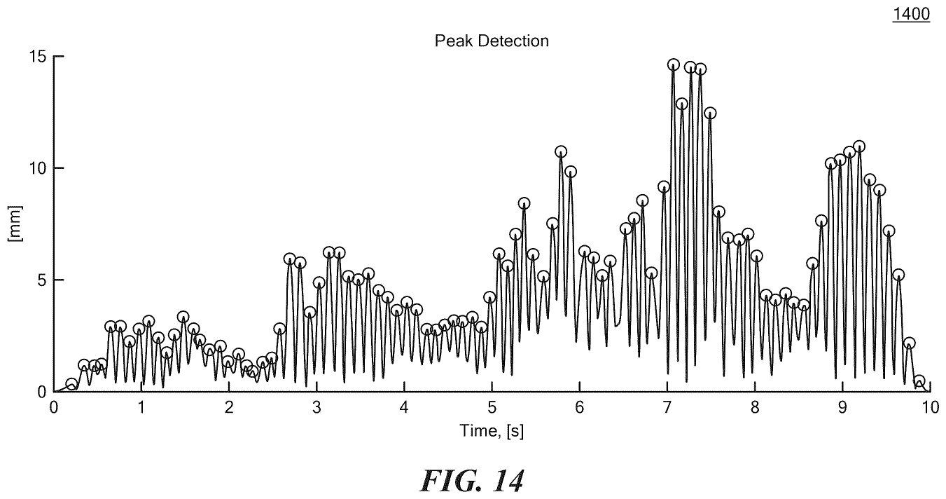

FIG. 14 is an example graph 1400 showing peaks of involuntary motions in accordance with various embodiments associated with, for example, block A11a, where, from the radii of the involuntary motion, peak detection is used to find peaks in the data which represent turning points, or extreme points, in the involuntary motions.

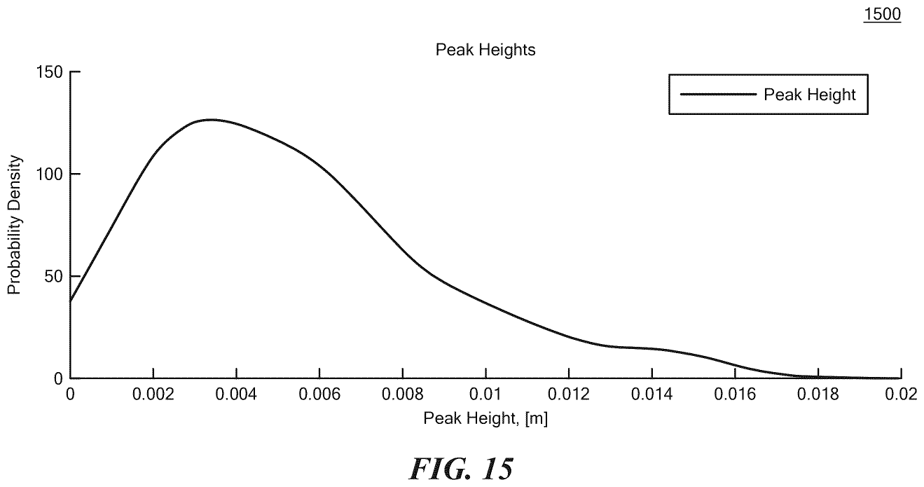

FIG. 15 is an example graph 1500 showing peak height probability of involuntary motions in accordance with various embodiments. FIG. 14 may be associated with block A12a, quantification version 1, where a Kernel Density Estimation is used on the collected radial peak heights which leads to a distribution describing the probability of seeing involuntary motions of a certain amplitude during the test.

Referring back to block A13a, a severity score is then constructed based on the probability distribution of the involuntary motions. It is constructed with the aim of describing the severity that a certain type of involuntary motions lead to, higher probability of larger involuntary motions gives a higher severity score. For example, in various embodiments, the severity score may calculated as the size if the involuntary motions at the 50% range+1.4*the 75% range+2*the 95% range weighing the tail parts of the distribution higher than the median. The value is then mapped onto a tanh function to have more granularity at the lower end and setting the range to give values from 0 to 100.

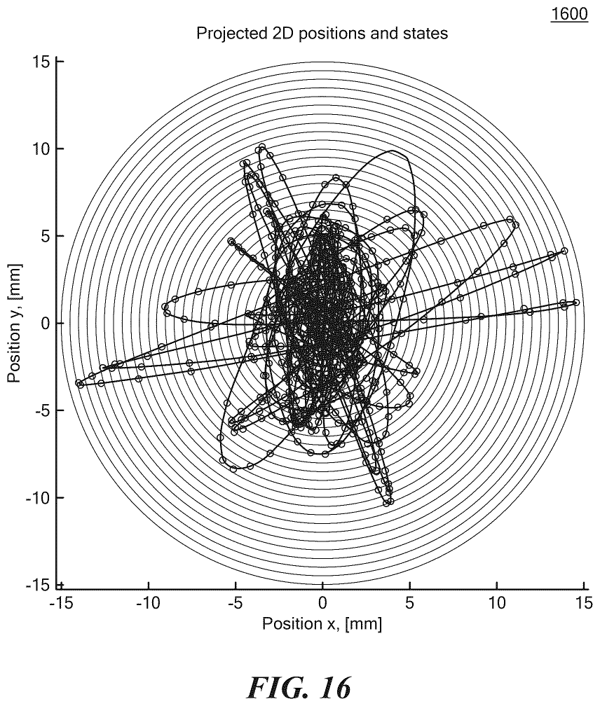

FIG. 16 is an example graph 1600 showing projected 2D positions and radii state in accordance with various embodiments. In various embodiments, FIG. 13 may be associated with block A11b, where suitable bin spacing is chosen for the radii of the involuntary motions. Each timestep is then looped over and at every step the state-to-state transition is counted to build up a transition matrix containing every transition from one radii state to another or within the same state.

Referring back to block A12b, quantification version 2, once the transition matrix is constructed it is checked if it is irreducible which in the context of the Markov chain model means that it is possible to visit and leave every state in the chain, (i.e. there are no states in which the system can become trapped). As mentioned above, if the transition matrix is reducible then the algorithm goes to step A13b otherwise it proceeds to step A14b.

Referring back to block A13b, quantification version 2, if the transition matrix is reducible, then a new slightly larger bin size is chosen for the involuntary movement radii and step A11b is redone.

Referring back to block A14b, quantification version 2, if the transition matrix is irreducible a stationary distribution of which state the radii system will be in over time is calculated by solving the eigenvalue equation of the transition matrix.

Referring back to block A15b, quantification version 2, a severity score is constructed from the stationary distribution attempting to describe the severity of the patients' symptoms. For example, in various embodiments, the score may be constructed as the mean of the distribution+2*the standard deviation+the 95% range, putting more emphasis on the tail of the distribution.

As mentioned above, block 4b describes the highly experimental quantification of the bradykinesia test. In various embodiments, this test does not focus on the amplitudes or the frequency of involuntary motions during the test but rather on the amplitudes and the frequencies of the voluntary motions performed during the test sequence and the main interest is in any interruptions or slowing of frequencies in the movements during the test.

For example, at block B1, from the sensor data the focus is on the gyroscope data since the patients are performing a turning motion back and forth. To find the main axes of rotation during the test a principal component analysis is performed on the gyroscope signal. The signal is then projected onto the principal axes and the largest component picked out. From this signal the median frequency is calculated.

At block B2, peak detection is used to find all peaks in the principal gyroscope signal above a specified limit rejecting some smaller peaks considered noise. In addition to the peak height the peak locations are also identified.

At block B3, using the peaks, and the peak locations a severity score is calculated. For example, in various embodiments, the severity score may be calculated as the (peak height IQR*0.5+peak frequency IQR)/(median peak height+median peak frequency*3). The aim is to reflect that a low spread in the peak height and frequency data leads to a lower score, so does a high median peak value and peak frequency, while the opposite is true if the spread is large, and the overall median values are low. The severity score along with the median frequency is sent back to the user.

As described above, at block D1, after a test has been performed on the mobile client the data is sent to an on premise database (on premise backend), once the data arrives it is first stored in a raw measurement database before a copy of the data is sent to the quantification algorithm.

At block D2, after a quantification has been made the results are stored in the on premise result database. It is from this database that the mobile client picks up the data associated with the tests that the user has performed.

The severity scores for all tests are meant to provide a simple and clear indication of the level of symptoms present during the performed test. All scores follow a principle of the lower the value the better but depending on the test the interpretation of the result is different. For all the tremor tests, (i.e. rest tremor, postural tremor, action tremor and the dyskinesia test), the score reflects a composite value of the probability of seeing involuntary motions of a certain amplitude weighted by an increasing amount for upper tail values. For the bradykinesia test the score represents a desire to keep the performed hand motions as large and as fast and as similar as possible during the test.

To use the scores to tune settings for (e.g. deep brain stimulation) the intention, in various embodiments, is to have the patient perform the desired tests, possibly representing the specific test or tests where the patient has the clearest symptoms, then have medical staff perform the desired tuning of the DBS settings and thereafter redo the test or tests and compare the scores, with lower scores indicating better settings.

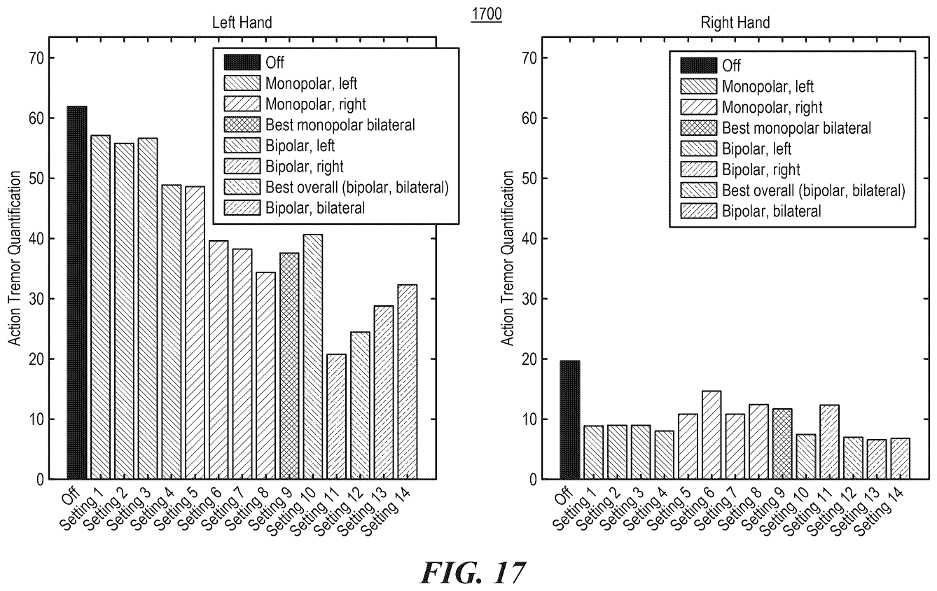

FIG. 17 is an example graph 2000 showing severity scores in accordance with various embodiments. In various embodiments, FIG. 17 shows an example of using an action tremor test to tune DBS settings for a patient. The figure shows the severity score for each of 14 different DBS settings and the off state. As shown in FIG. 17, the best setting might be considered to be setting 12 as it represents a good trade-off between symptom minimization for both the right and left hands for the patient.

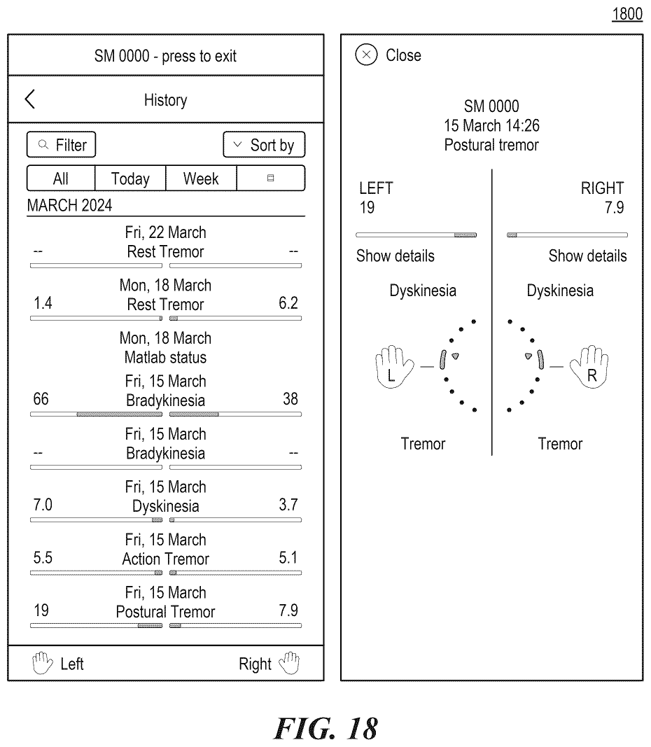

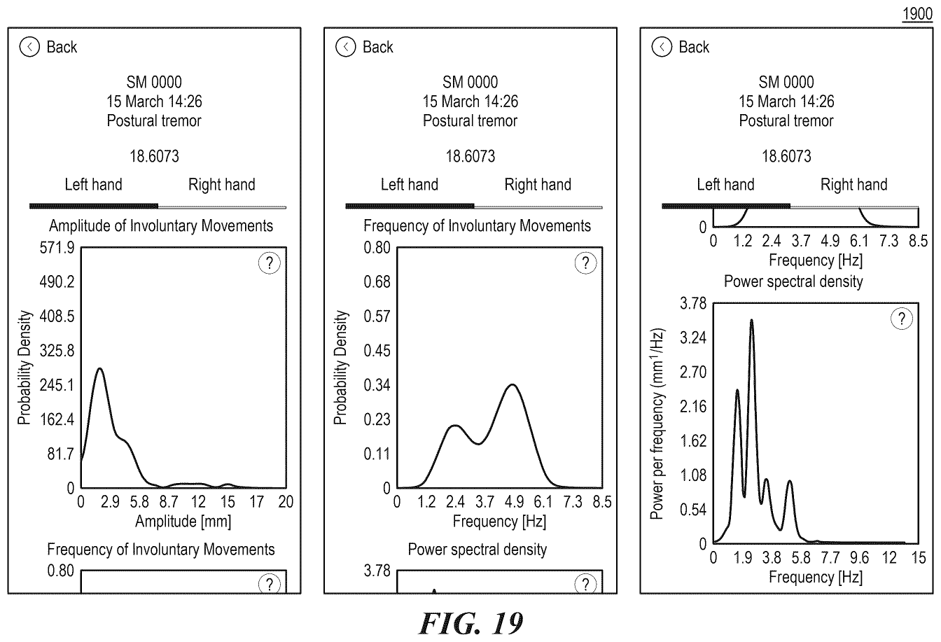

FIGS. 18-19 show results interfaces in accordance with various embodiments.

For example, FIG. 18 shows the base result screen 1800 where all measurements performed by the patient can be viewed, the base screen shows the type of measurement performed, the date it was performed and the severity score for both hands. The right image shows the measurement result view for a specific measurement after selecting it from the first list. It displays the severity score along with an indication whether the test showed signs of dyskinesias or tremor for both hands.

FIG. 19 shows the extended result view 1900 for a particular measurement which is accessible by clicking on the result in the individual result view. It displays in a single scrolling view details of the base signals that go into creating the severity score as well as some other information of interest. The results show the distribution of the peak heights of the involuntary motions and the frequency of the involuntary motions as well as the power spectral density of the projected 2D positions of the involuntary motions.

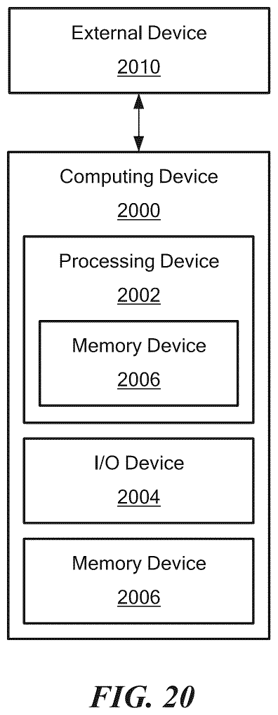

FIG. 20 shows an example computing device in accordance with various embodiments.

For example, FIG. 20 schematically shows a computing device 2000 in accordance with various embodiments. The computing device 2000 is one example of a computing device which is used to perform one or more operations of the process/method illustrated in FIG. 1. The computing device 2000 includes a processing device 2002, an input/output device 2004, and a memory device 2006. The computing device 2000 may be a stand-alone device, an embedded system, or a plurality of devices configured to perform the functions described with respect to one of the components of power network 100. Furthermore, the computing device 2000 may communicate with one or more external devices 2010. In various embodiments, the computing device 2000 may be the patient movement analysis system 150 of FIG. 1.

The input/output device 2004 enables the computing device 2000 to communicate with an external device 2010. For example, the input/output device 2004 may be a network adapter, a network credential, an interface, or a port (e.g., a USB port, serial port, parallel port, an analog port, a digital port, VGA, DVI, HDMI, FireWire, CAT 5, Ethernet, fiber, or any other type of port or interface), among other things. The input/output device 2004 may be comprised of hardware, software, or firmware. The input/output device 2004 may have more than one of these adapters, credentials, interfaces, or ports, such as a first port for receiving data and a second port for transmitting data, among other things.

The external device 2010 may be any type of device that allows data to be input or output from the computing device 2000. For example, the external device 2010 may be a meter, a control system, a sensor, a mobile device, a reader device, equipment, a handheld computer, a diagnostic tool, a controller, a computer, a server, a printer, a display, a visual indicator, a keyboard, a mouse, or a touch screen display, among other things. Furthermore, the external device 2010 may be integrated into the computing device 2000. More than one external device may be in communication with the computing device 2000. In various embodiments, the external device 2010 may be the patient device 100 of FIG. 1.

The processing device 2002 may be a programmable type, a dedicated, hardwired state machine, or a combination thereof. The processing device 2002 may further include multiple processors, Arithmetic-Logic Units (ALUs), Central Processing Units (CPUs), Digital Signal Processors (DSPs), or Field-programmable Gate Arrays (FPGA), among other things. For forms of the processing device 2002 with multiple processing units, distributed, pipelined, or parallel processing may be used. The processing device 2002 may be dedicated to performance of just the operations described herein or may be used in one or more additional applications. The processing device 2002 may be of a programmable variety that executes processes and processes data in accordance with programming instructions (such as software or firmware) stored in the memory device 2006. Alternatively or additionally, programming instructions are at least partially defined by hardwired logic or other hardware. The processing device 2002 may be comprised of one or more components of any type suitable to process the signals received from the input/output device 2004 or elsewhere, and provide desired output signals. Such components may include digital circuitry, analog circuitry, or a combination thereof.

The memory device 2006 in different embodiments may be of one or more types, such as a solid-state variety, electromagnetic variety, optical variety, or a combination of these forms, to name but a few examples. Furthermore, the memory device 2006 may be volatile, nonvolatile, transitory, non-transitory or a combination of these types, and some or all of the memory device 2006 may be of a portable variety, such as a disk, tape, memory stick, or cartridge, to name but a few examples. In addition, the memory device 2006 may store data which is manipulated by the processing device 2002, such as data representative of signals received from or sent to the input/output device 2004 in addition to or in lieu of storing programming instructions, among other things. As shown in FIG. 20, the memory device 2006 may be included with the processing device 2002 or coupled to the processing device 2002, but need not be included with both.

The operations and blocks of any method described above, such as of FIG. 2 are merely illustrative, and variations are contemplated to be within the scope of the present disclosure. In embodiments, the operations and blocks may include other operations not illustrated. In embodiments, the operations and blocks may not include every operation illustrated. In embodiments, the operations and blocks may be implemented in a different order than that illustrated. Such and other embodiments are contemplated to be within the scope of the present disclosure. Persons of skill in the art will appreciate that, although various example components may be described as performing various functions, operations and blocks, other components may perform those functions, operations and blocks described above.

It is contemplated that the various aspects, features, processes, and operations from the various embodiments may be used in any of the other embodiments unless expressly stated to the contrary. Certain operations illustrated may be implemented by a computer executing a computer program product on a non-transient, computer-readable storage medium, where the computer program product includes instructions causing the computer to execute one or more of the operations, or to issue commands to other devices to execute one or more operations.

While the present disclosure has been illustrated and described in detail in the drawings and foregoing description, the same is to be considered as illustrative and not restrictive in character, it being understood that only certain exemplary embodiments have been shown and described, and that all changes and modifications that come within the spirit of the present disclosure are desired to be protected. It should be understood that while the use of words such as “preferable,” “preferably,” “preferred” or “more preferred” utilized in the description above indicate that the feature so described may be more desirable, it nonetheless may not be necessary, and embodiments lacking the same may be contemplated as within the scope of the present disclosure, the scope being defined by the claims that follow. In reading the claims, it is intended that when words such as “a,” “an,” “at least one,” or “at least one portion” are used there is no intention to limit the claim to only one item unless specifically stated to the contrary in the claim. The term “of” may connote an association with, or a connection to, another item, as well as a belonging to, or a connection with, the other item as informed by the context in which it is used. The terms “coupled to,” “coupled with” and the like include indirect connection and coupling, and further include but do not require a direct coupling or connection unless expressly indicated to the contrary. When the language “at least a portion” or “a portion” is used, the item can include a portion or the entire item unless specifically stated to the contrary. Unless stated explicitly to the contrary, the terms “or” and “and/or” in a list of two or more list items may connote an individual list item, or a combination of list items. Unless stated explicitly to the contrary, the transitional term “having” is open-ended terminology, bearing the same meaning as the transitional term “comprising.”

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as a pre-configured, stand-alone hardware element and/or as preprogrammed hardware elements (e.g., application specific integrated circuits, FPGAs, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

The embodiments of the invention described above are intended to be merely exemplary; numerous variations and modifications will be apparent to those skilled in the art. Such variations and modifications are intended to be within the scope of the present invention as defined by any of the appended claims. It shall nevertheless be understood that no limitation of the scope of the present disclosure is hereby created, and that the present disclosure includes and protects such alterations, modifications, and further applications of the exemplary embodiments as would occur to one skilled in the art with the benefit of the present disclosure.

1. A method for quantifying movement patterns in a subject, comprising:
receiving motion data representing voluntary and involuntary movements of the subject from a sensor device worn or held by the subject;
extracting a signal indicative of involuntary movement deviations occurring in a plane diverging from a voluntary movement trajectory;
associating the extracted signal to a current position of the voluntary movement trajectory;
identifying peaks within the extracted signal, wherein each peak corresponds to a local maximum of involuntary movement magnitude;
estimating a probability density function (PDF) of the identified peaks;
computing a symptom severity score based on one or more quantiles of the estimated PDF; and
providing feedback to a user via a user interface based on the symptom severity score to adjust a parameter of a therapeutic device.

2. The method of claim 1, wherein the involuntary movement comprises at least one of tremor or dyskinesia.

3. The method of claim 1, wherein the PDF estimation includes using a kernel density estimation (KDE) process.

4. The method of claim 3, wherein the kernel density estimation uses a normal distribution as a kernel function.

5. The method of claim 4, wherein the kernel density estimation applies a bandwidth parameter determined by a maximum likelihood approach.

6. The method of claim 1, further comprising truncating the estimated PDF.

7. The method of claim 1, wherein identifying peaks comprises applying at least one of a threshold-based method, zero-crossing analysis, or derivative-based technique.

8. The method of claim 1, further comprising extracting statistical features from the identified peaks, including at least one of minimum peak magnitude, maximum peak magnitude, mean peak magnitude, median peak magnitude, standard deviation, or variance.

9. The method of claim 1, wherein the extracted statistical features are utilized in at least one of a clinical decision support system, an adaptive therapeutic device, or a machine learning algorithm for classification or prediction.

10. The method of claim 1, further comprising computing features from the identified peaks, wherein the features include at least one of a peak frequency, an inter-peak interval, or a peak clustering metric.

11. A system, comprising:
a sensor configured to detect motion data representing voluntary and involuntary movements of a subject;
a processor in communication with the sensor, the processor configured to receive, from the sensor, the motion data representing voluntary and involuntary movements of the subject, extract a signal indicative of involuntary movement deviations occurring in a plane diverging from a voluntary movement trajectory, associate the extracted signal to a current position of the voluntary movement trajectory, identify peaks within the extracted signal, wherein each peak corresponds to a local maximum of involuntary movement magnitude, estimate a probability density function (PDF) of the identified peaks, compute a symptom severity score based on one or more quantiles of the estimated PDF, and provide feedback to a user via a user interface based on the symptom severity score to adjust a parameter of a therapeutic device.

12. The system of claim 11, wherein the involuntary movement comprises at least one of tremor or dyskinesia.

13. The system of claim 11, wherein the PDF estimation includes using a kernel density estimation (KDE) process.

14. The system of claim 13, wherein the kernel density estimation uses a normal distribution as a kernel function.

15. The system of claim 14, wherein the processor is further configured to apply a bandwidth parameter determined by a maximum likelihood approach.

16. The system of claim 11, wherein the processor is further configured to truncate the estimated PDF.

17. The system of claim 11, wherein the processor is further configured to apply at least one of a threshold-based method, zero-crossing analysis, or derivative-based technique.

18. The system of claim 11, wherein the processor is further configured to extract statistical features from the identified peaks, including at least one of minimum peak magnitude, maximum peak magnitude, mean peak magnitude, median peak magnitude, standard deviation, or variance.

19. A computer program product for use on a computer system, the computer program product comprising a tangible, non-transient computer usable medium having computer readable program code thereon, the computer readable program code comprising:
program code for receiving motion data representing voluntary and involuntary movements of the subject from a sensor device worn or held by the subject;
program code for extracting a signal indicative of involuntary movement deviations occurring in a plane diverging from a voluntary movement trajectory;
program code for associating the extracted signal to a current position of the voluntary movement trajectory;
program code for identifying peaks within the extracted signal, wherein each peak corresponds to a local maximum of involuntary movement magnitude;
program code for estimating a probability density function (PDF) of the identified peaks;
program code for computing a symptom severity score based on one or more quantiles of the estimated PDF; and
program code for providing feedback to a user via a user interface based on the symptom severity score to adjust a parameter of a therapeutic device.

20. The computer product of claim 19, wherein the involuntary movement comprises at least one of tremor or dyskinesia.
